(19) United States
(12) Patent Application Publication
(10) Pub. No.: US 2023/0263423 A1
(43) Pub. Date: Aug. 24, 2023

(54) PROCESSING RECORDINGS OF A SUBJECT'S BREATHING

(71) Applicant: Respiri Limited, Melbourne, Victoria (AU)

(72) Inventors: Ronald Scott PLATT, Melbourne, Victoria (AU); Samaneh SARRAF SHIRAZI, Melbourne, Victoria (AU)

(21) Appl. No.: 17/755,670

(22) PCT Filed: Oct. 22, 2021
(86) PCT No.: PCT/AU2021/051232

§ 371 Date: May 4, 2022

(30) Foreign Application Priority Data
Oct. 22, 2020 (AU) 2020903832

(51) Int. Cl. A61B 5/08; A61B 5/00

(52) U.S. Cl. CPC A61B 5/0803; A61B 5/7221; A61B 5/725; A61B 5/7282

(57) ABSTRACT

A method (an corresponding system) of processing recordings of a subject's breathing, the method comprising: obtaining a tracheal recording of the subject's breathing; dividing the tracheal recording into a plurality of segments, and, for each segment of the plurality of segments: computing energy of the tracheal recording of the segment; computing power spectral density of the tracheal recording of the segment; processing the computed power spectral density to form a set of poles for the segment; iterating through the set of poles from a lowest frequency to a highest frequency to produce a set of pitches for the segment by discarding all poles outside a defined range around 1.0, and upon finding a pole having a magnitude within a defined range around 1.0, finding all other poles having a frequency that is a multiple of the frequency of the respective pole, and applying criteria to the segment to determine whether the segment is a candidate segment for being a wheeze in the tracheal recording, wherein the criteria include that a respective segment includes at least one pitch; and identifying each candidate segment that satisfies a proximity criterion in respect of at least one other candidate segment as being part of a wheeze in the tracheal recording.

### RELATED APPLICATION

This application claims convention priority from Australian provisional patent application no. 2020903832, entitled “PROCESSING RECORDINGS OF A SUBJECT'S BREATHING,” filed on 22 Oct. 2020, the entire content of which is herein incorporated by reference in its entirety for all purposes.

### FIELD

The invention related to processing recordings of a subject's breathing in order to identify breathing irregularities in the recordings.

### BACKGROUND

We have previously developed a system (currently marketed under the name “Wheezo”®) that comprises a hand-held recording device and associated software. The system is used by patients for long term management of their asthma. Patients use the recording device to make short, typically 30 second, recordings of their breathing sounds by placing the recording device on their trachea. The device communicates via Bluetooth with a smartphone running an application we have developed that transmits the recording to a centralized database infrastructure for analysis and future review. Software within the centralized database infrastructure is used to develop relationships between the sound recordings, medication usage and extraneous factors such as weather, air quality, pollen count, etc. These relationships provide information for patients to better understand what practices worsen or improve their baseline asthmatic condition. This information is provided to the patients via the smartphone application.

A component of the software infrastructure is an algorithm to analyze the breath sound recordings and detect “wheeze” which results from restriction of large and medium airways in the lungs. Wheeze is a key indicator of asthma so regular measurement of wheeze will reveal long-term changes in asthma. Physicians use a stethoscope placed on the chest or back to listen for wheeze sounds in a patient. Wheeze is heard in breathing sound as a whistling or singing like pitch. The analysis algorithm processes the sound signal and marks periods containing pitch that is characteristic of wheeze.

The recording device also has a second microphone for simultaneously recording ambient sound when measuring breathing. The recordings from the second microphone are used in order to distinguish between true wheeze and background noise. In particular, this second microphone enables periods with excessive extraneous background noise to be eliminated from analysis.

We have previously used subtraction to remove background sound from the tracheal recording. However, we have determined that this sometimes resulted in severe wheeze being cancelled out because severe wheezes tend to be picked up by the background microphone. As a result, severe wheeze would sometimes be missed by the wheeze detection algorithm.

There is a need for improved methods of monitoring of breathing.

### SUMMARY OF THE INVENTION

There is described a method of processing recordings of a subject's breathing, the method comprising:
obtaining a tracheal recording of the subject's breathing;
dividing the tracheal recording into a plurality of segments, and, for each segment of the plurality of segments:
   computing energy of the tracheal recording of the segment;
   computing power spectral density of the tracheal recording of the segment;
   processing the computed power spectral density to form a set of poles for the segment;
   iterating through the set of poles from a lowest frequency to a highest frequency to produce a set of pitches for the segment by discarding all poles outside a defined range around 1.0, and upon finding a pole having a magnitude within a defined range around 1.0, finding all other poles having a frequency that is a multiple of the frequency of the respective pole, and
   applying criteria to the segment to determine whether the segment is a candidate segment for being a wheeze in the tracheal recording, wherein the criteria include that a respective segment includes at least one pitch; and
identifying each candidate segment that satisfies a proximity criterion in respect of at least one other candidate segment as being part of a wheeze in the tracheal recording.

There is also described a system for monitoring a subject's breathing, the method comprising:
a recording device;
a processing module in communication with the recording device and configured to:
   receive at least a tracheal recording from the recording device;
   divide the tracheal recording into a plurality of segments, and, for each segment of the plurality of segments:
compute energy of the tracheal recording of the segment;
compute power spectral density of the tracheal recording of the segment;
process the computed power spectral density to form a set of poles for the segment;
iterate through the set of poles from a lowest frequency to a highest frequency to produce a set of pitches for the segment by discarding all poles outside a defined threshold of 1.0, and upon finding a pole having a magnitude within a defined threshold of 1.0, finding all other poles having a frequency that is a multiple of the frequency of the respective pole, and
apply criteria to the segment to determine whether the segment is a candidate segment for being a wheeze in the tracheal recording, wherein the criteria include that a respective segment includes at least one pitch; and
   identify each candidate segment that is satisfies a proximity criterion in respect of at least one other candidate segment as being part of a wheeze in the tracheal recording.

### BRIEF DESCRIPTION OF THE DRAWINGS

Embodiments of the diagram are described in relation to the accompanying figures in which:

FIG. 1 is a schematic diagram of a breathing recording system.

FIGS. 2A and 2B illustrate the occurrence of “wheeze” in breathing.

FIG. 3 shows poles plotted on the unit circle in the complex plane for wheeze and non-wheeze.

FIGS. 4A to 4C is a flow chart of an embodiment.

### DETAILED DESCRIPTION

Embodiments are described that provide a method of processing recordings of a subject's breathing that enables any wheeze to be identified within the recording.

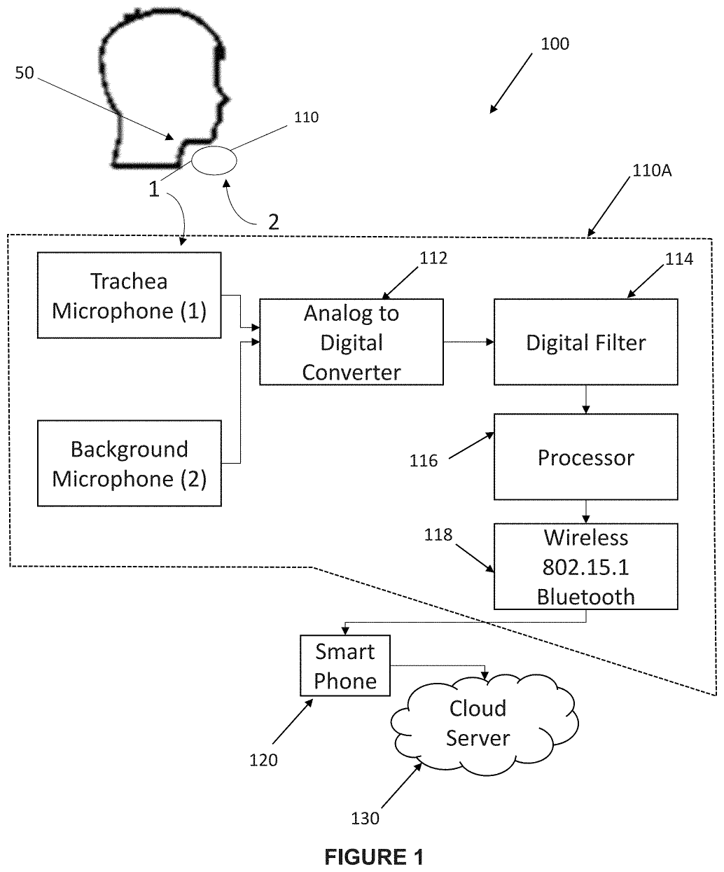

FIG. 1 is a schematic diagram of a recording system 100 being used to obtain a recording of a subject's breathing. As shown in FIG. 1, a recording device 110 is placed on the trachea 50 by the subject to obtain a short recording of breathing sounds. It will be appreciated that the recording device 110 may be used by someone else to obtain the recording, e.g. a physician.

The recording device 110 contains a tracheal microphone 1 that is intended to be placed adjacent to the trachea 50 by the patient and a background microphone 2 at the opposite end of the recording device 110 to record ambient sound. Some embodiments of the invention are directed at identifying wheeze within potentially noisy environments and include steps to mitigate against the effect of noise. The background microphone assists with implementation of such embodiments. In other embodiments, where recordings are captured in a quiet environment, other recording devices can be used such as a digital stethoscope or the microphone of a mobile device. In such embodiments, the sound recording will be primarily of the tracheal sound.

As shown by block diagram 110A of the recording device 110 has a processor 116 that responds to user activation of the recording device by pressing a button (not shown) to capture synchronized sound recordings from both tracheal 1 and background microphones (2). The captured sound recordings are passed through an analog to digital converter 112 and a digital filter 114 before being sent by processor 116 via Bluetooth transmitter 118 to the user's mobile device 120. The processor of the mobile device 120 executes an associated mobile device application that implements an analysis algorithm in order to analyse the sound recordings. The sound recordings are also transmitted via the mobile device application to a server 130 that hosts a centralized patient database.

In other examples, the analysis algorithm can be implemented somewhere different to at the mobile device, e.g. at the server 130 or within the recording device 110. The examples below are described in relation to an example, where the analysis is implemented at the mobile device.

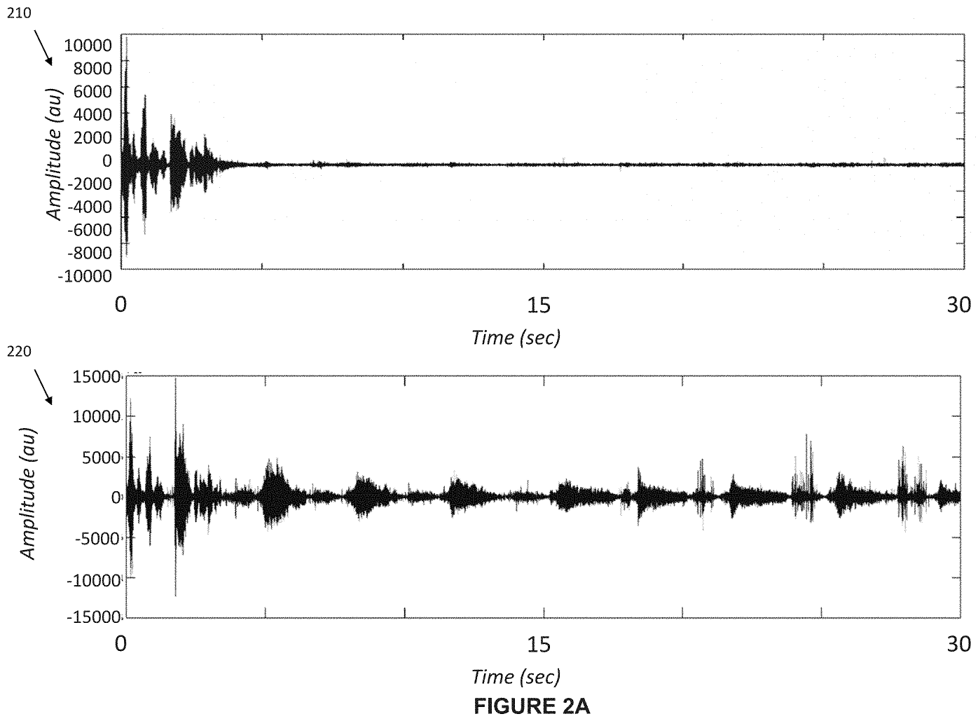

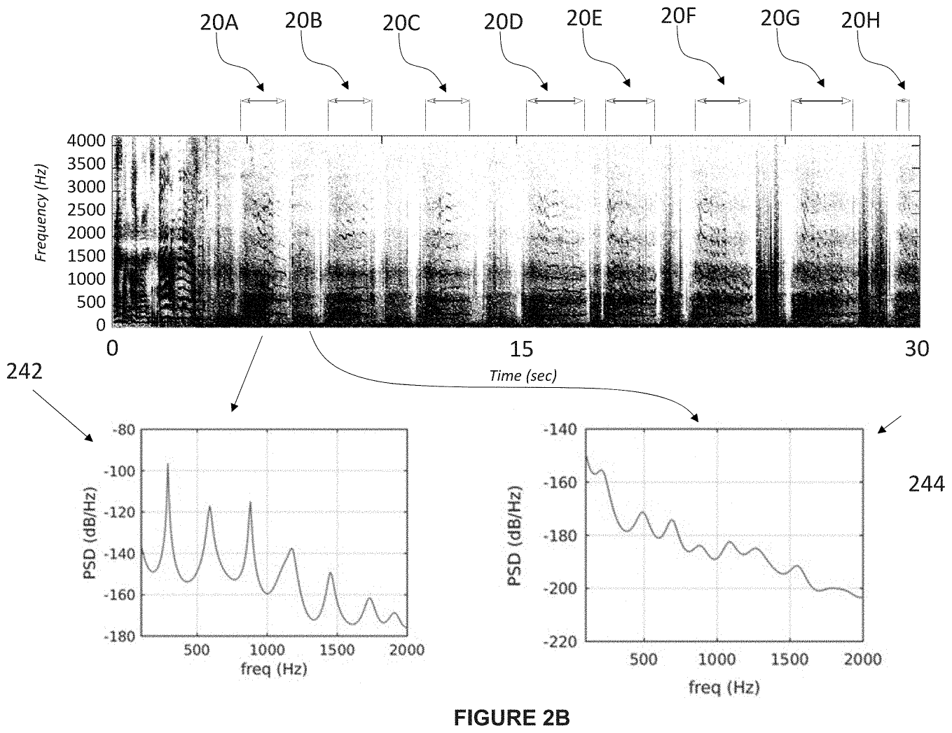

FIGS. 2A and 2B show graphs of representative 30 second recordings of tracheal sound 220 from top and background ambient sound 210 where the x-axis is time in seconds and the y-axis is amplitude (au). In these examples, the first 4 seconds of the recordings contain speech that is detected on both microphones 1,2. As described below, by comparing the power levels in the trachea and background signals, these first 4 seconds of recording are eliminated from analysis. The rest of the recording is suitable for wheeze analysis.

In the tracheal recording, eight breath cycles can be seen as the waxing and waning of the sound amplitude. The frequency composition or spectrogram 230 of the tracheal recording is shown below the sound signals in the figure. The spectrogram shows darker areas at frequencies that dominate the recording. This tracheal recording 220 contains significant wheeze throughout the expiratory phase of each breath cycle. By listening to the recording 220 and simultaneously viewing the spectrogram 230, portions 20A-20H containing wheeze sound were identified and their periods of duration indicated by arrows above the spectrogram 230.

Wheeze sound is a clear pitch composed of a fundamental frequency plus a set of harmonic frequencies. The harmonic frequencies are integer multiples of the fundamental frequency. Within the wheeze portions of the recording, sets of equally spaced lines are visible in the spectrogram. The lowest line is the fundamental frequency with the lines above being the harmonics. As the frequency of the pitch changes, the entire set of lines moves together. Portions without wheeze do not contain clear pitch and therefore do not contain these lines.

Power spectrum estimates 242, 244 of very short segments of the sound recording show the difference between wheeze 242 and non-wheeze 244 as shown at the bottom of the figure. In the segment 20A containing wheeze, the fundamental frequency and its harmonics are clearly evident in the power spectrum 242 and the peaks are equally spaced. Conversely, the power spectrum 244 of a segment without wheeze (between wheeze segments 20A and 20B) shows no clear harmonic content. That is, normal breathing normally has a broad, mainly uniform spectrum, while wheeze includes resonance arising from one or more constrictions in the airway caused by an underlying condition such as asthma.

Disclosed embodiments work by identifying which portions of the recording contain clear pitch. In an embodiment, the algorithm uses an auto-regressive (AR) power spectrum estimation as part of identifying pitch.

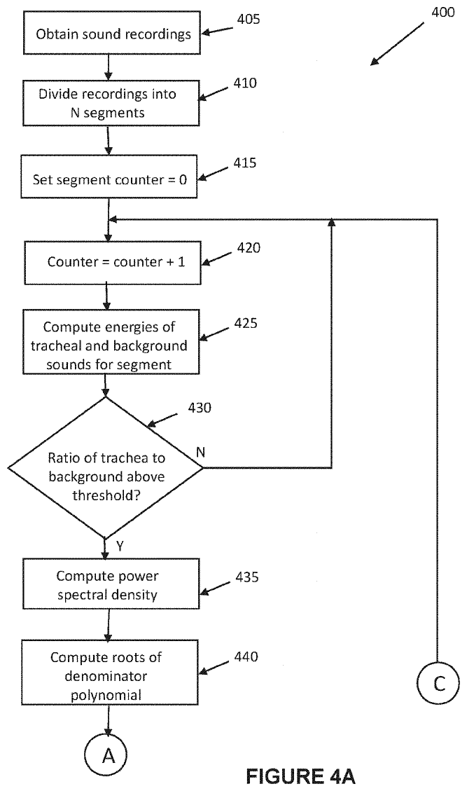

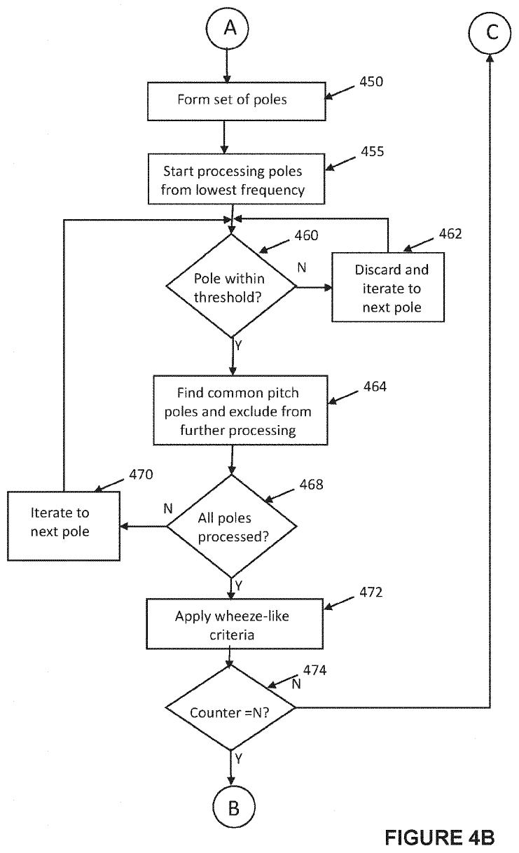

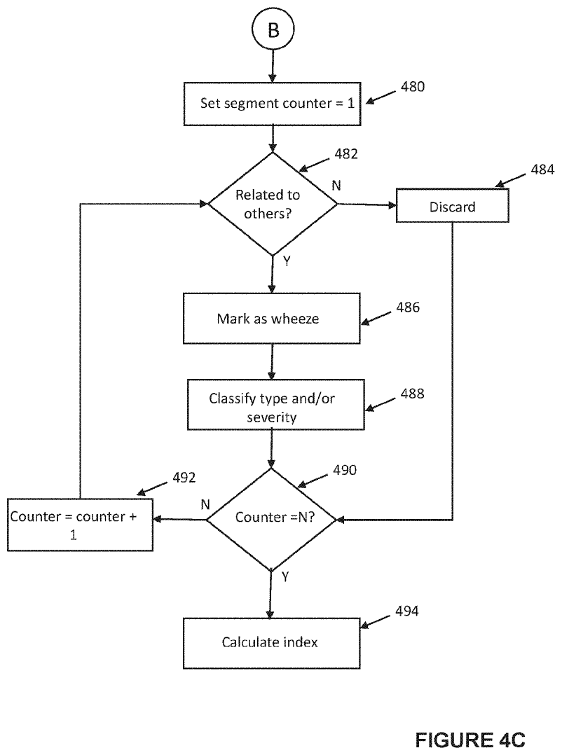

FIG. 4 is a flow chart 400 of an embodiment of an analysis method performed when a processor of the mobile device 120 executed the mobile device application. At step 405, the mobile device application receives data from the recording device 110 that includes recordings from the tracheal 1 and background 2 microphones. That is, the method of FIG. 4 is intended for use in potentially noisy environments.

At step 410, the method involves dividing the tracheal and background sound recordings into N segments, in this example 50 msec segments so that for a thirty second recording there are 600 segments. (Only the tracheal recording need be processed in quiet environments.) In this example, a sampling rate of 10800 cycles per second is used. As a result, each segment contains 540 samples. In this example, the resultant data is stored as signed 16-bit integers in the memory of the mobile device.

It will be apparent to the person skilled in the art, that the recording length, sampling rate and segment length may vary depending on the implementation. Further, in some examples, overlapping segments may be employed in order to better detect transients in the recordings.

At step 415, the method involves initializing a counter by initially setting the counter to zero. The counter is incremented by one at step 420, so that a first of two iterative loops through all of the segments starts by processing the first segment. Persons skilled in the art will appreciate that other processing orders are possible, particularly where the recordings are short. However, processing the segments in chronological order starting at segment 1 and finishing with segment N is advantageous for real-time processing.

At step 425, for each segment, the energy of the trachea and background sound is computed by the processor as the standard deviation. At step 430 it is determined whether the ratio of tracheal sound to background sound is above a threshold. In this example, the criterion is that only segments where the energy in the tracheal signal have more than a defined multiple of 2.2 times as the energy in background signal are used. This criterion ensures that there is enough tracheal signal relative to background signal and hence omits, for example, segments that capture the subject speaking.

In this example, segments where the ratio of tracheal sound to background sound is less than the threshold are not processed further for computational efficiency. That is, the method reverts to step 420, increments the counter and hence at step 425 computes the energies for the next segment. However, in other examples, this criterion could be applied later, for example when other criteria are applied to the segments to determine whether they are candidates for wheeze.

When a segment is processed further, at step 435, power spectral density is computed by the processor as, for example, a 64 or 96 order auto-regressive (AR) model. In this implementation, the calculation is performed with a 64-order model for computational efficiency using the standard public domain Burg method described in Burg JP, “The relationship between maximum entropy spectra and maximum likelihood spectra”, Geophysics, 1972 April; 37(2):375-6. Persons skilled in the art will appreciate that other methods of computing the AR model could be used such as the Yule-Walker method or a least-squares approach. In addition, future embodiments of the algorithm may use a more generalized auto-regressive moving average (ARMA) models to quantify the spectral distribution of the signal. The order of the spectral estimates can be adjusted in other examples.

The auto-regressive model is the optimized regression of a signal onto previous values of itself. The computed model defines the coefficients a₁ through aₚ where p is the order of the model as shown in Eq. 1. The term w (n) represents residual uncorrelated error.

Y(n)=a₁Y(n−1)+a₂Y(n−2)+ . . . +aₚY(n−p)+w(n) Eq. 1

In the frequency domain, the auto-regressive model can be represented as a filter or transfer function in which the coefficients define a polynomial in the denominator as shown in Eq. 2. The power spectrum magnitude is computed by evaluating the transfer function at values of complex variable z on the unit circle at angles ranging from 0 to π which correspond to frequencies of 0 to half the digital sampling rate.

$$P ( z ) = \frac{1}{1 - {a}_{1} z - {a}_{2} {z}^{2} - … - {a}_{p} {z}^{p}} \qquad Eq . 2$$

Values of z for which the denominator of the transfer function becomes zero are called the poles of the transfer function. The poles correspond to frequencies that dominate the frequency content of the signal.

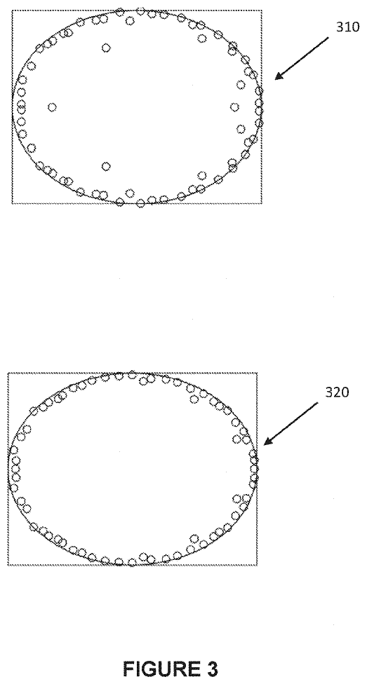

FIG. 3 shows the poles plotted on the unit circle in the complex plane of the AR models during examples of breathing recordings corresponding to wheeze 310 and non-wheeze 320 These plots 310,320 correspond to power spectra 242 and 244 respectively shown in FIG. 2. A frequency of zero is on the right and following the circle counter clockwise to the left side corresponds to a frequency of half the sample rate. In the non-wheeze plot 320, all of the poles are consistently a small distance off of the unit circle which results in relatively uniform evaluation of the transfer function and a relatively flat power spectrum.

Conversely, the poles on the plot 310 during wheeze are staggered with some almost on the unit circle and others quite far off. This causes the transfer function to fluctuate between low and high values and results in peaks in the power spectrum. The poles that are closest to the unit circle are the peaks of the power spectrum and the height of each peak is inversely proportional to the distance between the pole and the unit circle.

In the embodiments, pitch is identified using peak analysis that involves finding groups of peaks with frequencies that are harmonically related. The pitch detection portion of algorithm operates by processing the roots of the denominator polynomial. In the embodiments, this algorithm allows finding multiple pitches.

At step 440, the method computes the roots of the denominator polynomial using a numerical method.

At step 450, a set of poles is formed by the processor for the respective segment by retaining only poles with positive complex components and sorting in ascending order by frequency. Only poles with positive complex components are retained because the poles with negative complex components are mirror images of those with positive complex roots and are redundant for the purpose of pitch detection.

Beginning at step 455, the list of poles is processed sequentially from lowest to highest frequency. At step 460, the processor determines whether the current pole being processed has a magnitude within a defined range around 1.0, in this example plus or minus 0.5 percent of 1.0, If not, at step 462 the pole is discarded from the set and the method iterates to determining whether the next pole is within the range. If at step 460, the pole is within the range, all poles above having a frequency that is a multiple within 2 percent of the fundamental are marked by the processor as being part of a common pitch at step 464 and excluded from further processing.

At step 469, the processor determines whether all poles have been processed and if not iterates to the next pole at step 470 (skipping over any poles already marked as part of a pitch). When the processor determines at step 472 that all poles are processed, the result is a list (set) of unique pitches and the number of harmonics that compose each pitch stored in the memory of the mobile device. The fundamental frequency of each pitch is also retained and available later for further use.

Wheeze Categorization

Having computed the power and pitch content of each segment, at step 472, the method includes a categorization process involving applying a set of criteria to each segment in order to identify whether each segment is (or isn't) “wheeze-like”. That is, a candidate for being treated as wheeze if it can be related to one or more other wheeze-like segments as described below. In the embodiment, the criteria are that are applied are strict “in” or “out criteria. However, the skilled person will appreciate that in other embodiments, criteria may be weighted relative to one another.

A first criterion is that the segment doesn't saturate the input range of the microphone. Under this criterion, segments having a standard deviation more than 12000, are discarded.

A second criterion for a segment to be considered wheeze-like is for it to contain at least one pitch. In other embodiments more harmonics could be employed with resulting great specificity but potentially lower sensitivity.

A third criterion is that the one pitch has a fundamental in a defined range, in this example, between 100 and 1200 Hz.

The algorithm treats wheeze-like segments as being part of a true wheeze episode if they satisfy a proximity criterion of occurring near other wheeze-like segments. In this example, if there are at least two consecutive wheeze-like segments. This is because wheeze has to be at least 100 ms long and the algorithm uses a 50 ms sampling window. Accordingly, after each segment has been categorized as wheeze-like (or not), the application running on the mobile device starts a further iterative loop to seek to group them into episodes of wheeze.

In this example, the method iterates chronologically through the segments again and accordingly at step 480 the segment counter is set to 1.

At step 482, the processor determines whether a wheeze like segment is related to at least one other segment, in this example each segment is marked as wheeze at step 486 if there are other wheeze segments either immediately before or after and if not, it is discarded at step 484.

In other embodiments, other techniques could be used to identify groups of wheeze-like segments, for example, by looking two segments forward and back or using speech recognition techniques to find nearby matching pitches.

In some examples, it may be desired to sub-classify wheeze episodes. FIG. 4, illustrates that in other examples each wheeze may be sub classified by type and/or severity at step 488. In this respect, the sound recordings include sounds ranging from low frequency gurgling sounds at around 100 Hz to high pitched sounds in the order of 1500 Hz. Physicians are able to distinguish between various types of disorders such as stridor, vocal cord dysfunction, lower respiratory and high respiratory wheeze, and wheeze severity by listening to such sounds. For example, low frequency sounds are more likely to be linked to the upper respiratory tract and likely to be indicative of less-severe wheeze. Accordingly, classifying the segment as being a low frequency segment and including this in the output of the system 100 may assist a person reviewing the output to reach a diagnosis. In other examples, further data such as features of pitch and variation in pitch to can be used to further define the distinction between various disorders to provide a more specific classification of the segment.

At step 490, the processor determines whether this is the last wheeze-like segment, and if not increments the counter by one at step 492 so that the next segment will be processed. When all wheeze-like segments have been processed, the method has identified all wheeze episodes in the tracheal recording. In some examples, the identified wheeze can be output to the subject, or can be stored to server for review by a physician who has access to server. In some examples, the system 100 can output the identified wheeze by using the identified segments to place markers on a spectrogram such as shown in FIG. 2. Other data such as the segment classifications outlined above could also be overlayed on such a spectrogram.

Wheeze Index

After all segments are processed, at step 494, a wheeze index is calculated as the number of wheeze segments over the total number of segments that had acceptable tracheal signal energy—that is sufficiently more tracheal energy than the background recording as described above. The wheeze index is output on the display of the mobile device by the mobile application to provide feedback to the subject. The wheeze index can also be sent to server 130. The skilled person will appreciate that feedback could be provided in other forms, for example, messages indicative of whether the wheeze is normal and/or indicative of the subject needing to seek medical attention.

The processor 116 can operate responsive to program code stored in an operably interfaced memory as a computer program, such as a mobile application, in order to implement the functionality of the processor 116 as herein described. The computer program can generally be stored in any suitably computer readable storage medium, for immediate access by the processor 116 or for access after copying said computer program into a memory workspace accessible to the processor 116.

While the invention has been described with respect to the figures, it will be appreciated that many modifications and changes may be made by those skilled in the art without departing from the spirit of the invention. Any variation and derivation from the above description and figures are included in the scope of the present invention as defined by the claims.

1. A method of processing recordings of a subject's breathing, the method comprising:
obtaining a tracheal recording of the subject's breathing;
dividing the tracheal recording into a plurality of segments, and, for each segment of the plurality of segments:
computing energy of the tracheal recording of the segment;
computing power spectral density of the tracheal recording of the segment;
processing the computed power spectral density to form a set of poles for the segment;
iterating through the set of poles from a lowest frequency to a highest frequency to produce a set of pitches for the segment by discarding all poles outside a defined range around 1.0, and upon finding a pole having a magnitude within a defined range around 1.0, finding all other poles having a frequency that is a multiple of the frequency of the respective pole, and
applying criteria to the segment to determine whether the segment is a candidate segment for being a wheeze in the tracheal recording, wherein the criteria include that a respective segment includes at least one pitch; and
identifying each candidate segment that satisfies a proximity criterion in respect of at least one other candidate segment as being part of a wheeze in the tracheal recording.

2. The method of claim 1, comprising:
obtaining a background recording of the subject's breathing that is concurrent with the tracheal recording;
dividing the background recording into a plurality of segments, each corresponding to a segment of the tracheal recording, and, for each segment of the plurality of segments of the background recording, computing energy of the background recording; and
upon the computed tracheal energy of a segment of the tracheal recording being less than a defined multiple of the background energy of a concurrent segment of the background recording, treating the tracheal segment as not being a candidate for being part of a wheeze.

3. The method of claim 1, wherein the power spectral density is computed as an auto-regressive model.

4. The method of claim 3, wherein the auto-regressive model is a 64-order model.

5. The method of claim 3, wherein the auto-regressive model is computed using the Burg method.

6. The method of claim 3, comprising:
determining a set of coefficients from the equation
Y(n)=a₁Y(n−1)+a₂Y(n−2)+ . . . +aₚY(n−p)+w(n)
where a₁ through aₚ are the coefficients, p is the order of the model, and w(n) represents residual uncorrelated error; and
determining values of z for which the denominator of a transfer function becomes zero, wherein the transfer function is
$$P ( z ) = \frac{1}{1 - {a}_{1} z - {a}_{2} {z}^{2} - … - {a}_{p} {z}^{p}}$$

7. The method of claim 6, wherein processing the computed power spectral density to form a set of poles for the segment comprises retaining only poles with positive complex roots.

8. The method of claim 1, wherein the applied criteria include the segment not saturating the input range of a tracheal microphone used to capture the tracheal recording.

9. The method of claim 1, wherein the applied criteria include that at least one pitch in the segment has a fundamental frequency in a defined range.

10. The method of claim 1, comprising calculating an index as a number of wheeze segments over the total number of segments with tracheal signal energy that are a defined multiple of the background energy of the segment.

11. A system for monitoring a subject's breathing, the method comprising:
a recording device; and
a processing module in communication with the recording device and configured to:
receive at least a tracheal recording from the recording device;
divide the tracheal recording into a plurality of segments, and, for each segment of the plurality of segments:
compute energy of the tracheal recording of the segment;
compute power spectral density of the tracheal recording of the segment;
process the computed power spectral density to form a set of poles for the segment;
iterate through the set of poles from a lowest frequency to a highest frequency to produce a set of pitches for the segment by discarding all poles outside a defined threshold of 1.0, and upon finding a pole having a magnitude within a defined threshold of 1.0, finding all other poles having a frequency that is a multiple of the frequency of the respective pole, and
apply criteria to the segment to determine whether the segment is a candidate segment for being a wheeze in the tracheal recording, wherein the criteria include that a respective segment includes at least one pitch; and
identify each candidate segment that is satisfies a proximity criterion in respect of at least one other candidate segment as being part of a wheeze in the tracheal recording.

12. The system of claim 11, wherein the recording device comprises a tracheal microphone and a background microphone, and the recording device is configured to obtain concurrent tracheal and background recordings of the subject's breathing, and wherein the processing module is further configured to:
receive a background recording of the subject's breathing that is concurrent with the tracheal recording;
divide the background recording into a plurality of segments, each corresponding to a segment of the tracheal recording, and, for each segment of the plurality of segments of the background recording, compute energy of the background recording; and
upon the computed tracheal energy of a segment of the tracheal recording being less than a defined multiple of the background energy of a concurrent segment of the background recording, treat the tracheal segment as not being a candidate for being part of a wheeze.

13. (canceled)

14. A computer readable storage medium storing a computer program comprising executable program code configured to cause a processor to implement the method of claim 1.
